(19) United States
(12) Patent Application Publication
(10) Pub. No.: US 2021/0346428 A1
(43) Pub. Date: Nov. 11, 2021

(54) BIOAVAILABLE MINERALS FOR THE MITIGATION OF CORONA VIRUSES (COVID 19), MANUFACTURING METHOD AND A TREATMENT METHOD

(71) Applicant: Paul R. Simpson, Granite Falls, WA (US)

(72) Inventor: Paul R. Simpson, Granite Falls, WA (US)

(21) Appl. No.: 17/245,085

(22) Filed: Apr. 30, 2021

(30) Foreign Application Priority Data
May 6, 2020 (AU) 2020901434

(51) Int. Cl. A61K 33/34; A61K 33/26; A61K 33/30; A61K 33/32; A61K 33/04; A61K 47/02; A61K 47/14; A61K 9/00

(52) U.S. Cl. CPC A61K 33/34; A61K 33/26; A61K 33/30; A61K 9/0014; A61K 33/04; A61K 47/02; A61K 47/14; A61K 33/32

(57) ABSTRACT

The present invention relates to animals including humans with mineral deficiencies that become viable to corona virus attacks. An improved method of treating mineral deficiencies is therefore desired. This invention is employing bioavailable minerals in a liquid complex that are pharmaceutically acceptable carriers including water. The composition for use in the method may include supporting animals including human nutrients such as botanical extracts, human hormones, vitamins, and mineral supplements necessary for animals including human health.

### FIELD OF THE INVENTION

The present invention relates to a composition which includes bioavailable minerals for the mitigation of viruses in creatures, especially in animal including human, a manufacturing method of the composition, and an improved method of treating mineral deficiencies.

### BACKGROUND OF THE INVENTION

Animals including human have mechanisms to contain and or eliminate attacks from viruses. They utilize a variety of methods to inhibit the development of viruses. are subject to a variety of chemical changes that an animal including human may utilize to discourage the viruses from replicating and leading to their consequential demise.

An early example is Man's use of salt to preserve food, the principle in the chemical composition is sodium, a metal.

An animal including human with adequate mineral availability will adjust the transport mechanisms within the animal including human to mitigate attacks from viruses. These are accomplished with REDOX (reduction-oxidation) and measured by the balance of pH & energy within the animal including human including human. FIG. 4 illustrates REDOX the relationship of Electrical Charge (Eh) to Acid-Alkalinity (pH). To generate energy, an animal including human requires minerals. At least Two dissimilar minerals in an electrolyte generate 1.5 volts and when cells operate in series, higher voltages. Through electrolysis an animal including human can separate minerals from compounds sufficient to maintain their energy requirements and an ideal REDOX of Eh4.5—pH6.50. This varies with each animal including human and the conditions where it grows.

Other mechanisms active within an animal including human are SOD (Superoxide Dismutase) and ROS (Reactive Oxygen Species) which require energy from mineral participation. Three metals for optimum frequency modulated power for the conversion of O3 to hydrogen peroxide.

Creatures, especially animal including humans, can suffer from mineral deficiencies that result in undesirable states, including attacks from viruses A variety of methods are known for treating these undesirable states,

but none have been entirely satisfactory. An improved method for treating mineral deficiencies is therefore desired. The discussion above is merely provided for general background information and not intended to be used as an aid in determining the scope of the claimed subject matters.

Studies have shown that many skin abnormalities are caused by the presence of non-typical or mutated cells. By eliminating non-typical or mutated anaerobic cells and latent toxicity, growth of new, healthy, aerobic cells is facilitated.

### SUMMARY OF THE INVENTION

In one aspect of the invention there is proposed a composition for treating mineral deficiencies, comprising: at least one complex cation and inorganic coordination complexes formed by the coordinate bond formation between an electropositive mineral cation and molecular groups; the inorganic coordination complexes including a protective cation mineral ligand carrier, that specifically releases the mineral cation to supply areas that require such a mineral; wherein the complex cation and inorganic coordination complexes is absorbed by diffusion and the mineral cation can then move freely throughout the internal animal including human or human circulation, without the cation binding to targets that do not have mineral deficiency for this cation.

In preference the ligand cation complex is ammonia or super-oxide dismutase.

In preference the ligand cation complex is ammonium hydrogen sulphate ((NH₄) HSO₄).

In preference the complex cation is mineral cation.

In preference the mineral cation is zinc or/and copper cation.

In preference the composition further comprises one of manganese, magnesium, cobalt, chromium, molybdenum, selenium, and vanadium.

In preference the composition further comprises one of botanical extracts, animal including human hormones, vitamins, and mineral supplements necessary for animal including human health.

In preference the ratio of zinc to copper of the composition is 7:2.

In preference the composition further comprises the composition has a pH of less than 1.0.

In a further aspect of the invention there is proposed a method of manufacturing a composition for treating mineral deficiencies of claim 1, comprising: preparing a solution of ammonium hydrogen sulphate ((NH4)HSO₄) using sulfuric acid (H₂SO₄); diluting the ammonium hydrogen sulphate with water to form a mixture;

adding sulfuric acid;
adding a solution iron sulphate (FeSO4) and water; and/or
adding a solution manganese sulphate (MgSO4) and water; and/or
adding a solution zinc sulphate (ZnSO4) and water; and/or
adding a solution copper sulphate (CuSO4) and water; and
agitating the mixture until the ammonium hydrogen sulphate, the sulfuric acid, the water, the manganese sulphate, the iron sulphate, the copper sulphate and the zinc sulphate are blended together to form a zinc metal-ligand complex comprising a first plurality of ammonia ligands and a copper metal-ligand complex comprising a second plurality of ammonia ligands.

In preference the method further comprises; adding at least one delivery agent to the composition prior to the step of treating.

In preference the method of treating mineral deficiencies comprises treating a creature with a composition, the composition being formed by the above.

In preference the creature is an animal including human.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will refer to several drawings as follows.

FIG. 1 is a drawing of a typical cell structure;

FIG. 2 is a drawing of the KREBS CYCLE of cells;

FIG. 3 is a drawing of the body requirement for minerals for conversion of Super Dismutase (SOD);

FIG. 4 is a table depicting REDOX the relationship of Electrical Charge (Eh) to Acid-Alkalinity (pH);

FIG. 5 is the production flow chart of the composition according to an embodiment of the present invention.

### DESCRIPTION OF THE INVENTION

The following detailed description of the invention refers to the accompanying drawings. Wherever possible, the same reference numbers will be used throughout the drawings and the following description to refer to the same and like parts. Dimensions of certain parts shown in the drawings may have been modified and/or exaggerated for the purposes of clarity or illustration.

This invention proposes employing bioavailable minerals in a liquid complex that are pharmaceutically acceptable carriers including water which is used for treating mineral deficiencies in animal including humans, such as spray including nasal spray and/or nebulizer, mists, injection, rinse or oral. Moreover, the bioavailable minerals can be included in formula drugs or even in the form of gels, agars or dermal or transdermal patches. Consequently, a composition including a cation and a cation ligand carrier is formed which can increase productivity and reduce disease severity and insect damage in animal including humans.

Element Zinc is essential to healthy immune function especially in the various skin layers, recognized as a possible treatment for cancers, powerful anti-inflammatory and shown to disrupt viral activity both inside and outside the cell. Copper utilized in the mitochondrial membrane generates most of the energy needed to power biochemical reactions. High concentration levels of Copper and Zinc are toxic to abnormal cells causing normal, programmed cell death. Zinc, Copper and Silver Dihydrogen Citrate are toxic to pathogenic microbes.

Transition metals manganese (Mn), iron (Fe), cobalt (Co), nickel (Ni), copper (Cu), and zinc (Zn) found in the D-block of the periodic table are present in biological systems as metal complexes. These ions are able to form coordinate covalent bonds with species called ligands. These ligands have atoms that have electron pairs that are available to be shared with metallic ions that have a positive charge and empty coordination sphere orbitals. Most organic molecules and ions that have oxygen and nitrogen will have these paired electrons available for bonding with metal ions. When a metal ion forms a coordinate bond with a ligand, a complex is formed. When this complex has a net charge, it is a complex ion. Neutral complexes will not be soluble in the aqueous environment of cells. Depending on the charge of the metal ion and ligand, both positive and negative complexes can be formed, and will be more soluble in biological environs.

There is an equilibrium established in an organism when a complex is present. Depending on the strength of the bond between the ligand and metal ion, a small amount of the free metal ion will be present. This equilibrium is pH dependent and also dependent on other ligands that may be present in the organism.

The efficiency of the composition of the present invention is directly proportional to the amount of free metal ions, for instance zinc ions, copper ions or their sum. Sub nano-molar concentrations of zinc would exist as an aqua-complex, i.e., complexed by water molecules. In some embodiments, the metal ions of the compositions are zinc which is complexed with amine through the dermis where it is picked up by zinc transport proteins and other naturally occurring ligands and transported to the cell. Then, topical application of zinc will lead to a localized higher concentration of free zinc and enhance absorption into the surrounding tissue which therefore treats diseases because of mineral deficiencies.

An embodiment of the present invention provides topical products composed of a proprietary mineral complex formulated to regenerate healthy cellular function. This unique mineral complex which contains ionic copper-zinc enhances new cell growth with a nutrient rich combination of vitamins that synergistically control and eliminate viral, bacterial, and fungal infections. First and foremost, the principles of the biology working mechanism of the compositions or products provided by the present invention and the reasons why it can treat, improve, or cure viruses and regenerate cell in animal including human or human bodies are going to be discussed.

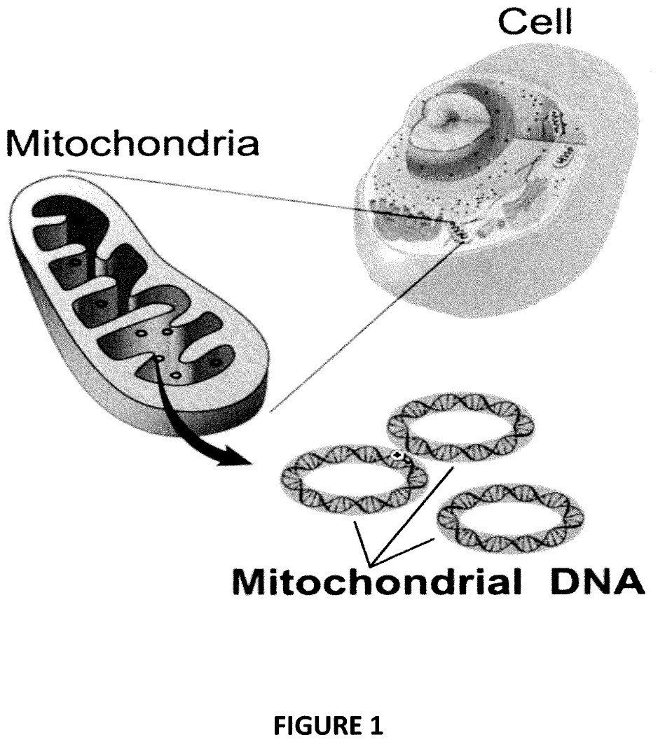

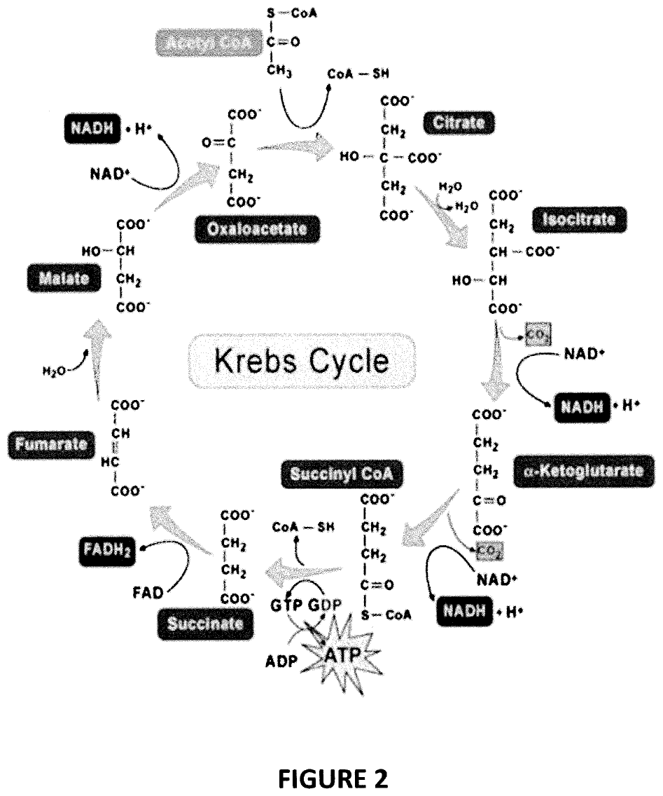

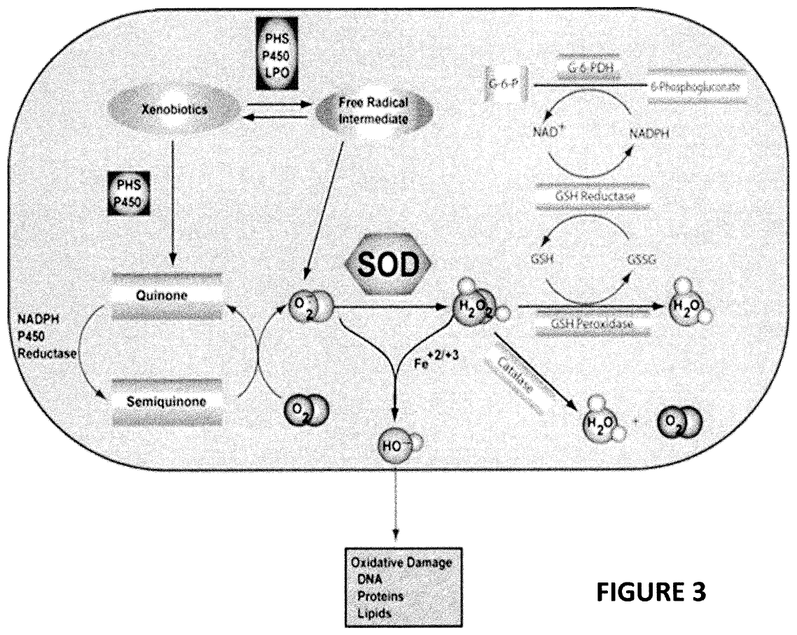

On one hand, healthy cells, as shown in FIG. 1, require energy to survive. Carbs, fats and proteins are eventually broken down into two carbon units (Acetyl-CoA) passing through the Krebs Cycle as shown in FIG. 2 to become electrons tied up in NADH and FADH₂ and available for pumping electrons into the cell inter-membrane, and to be further transformed to the inter-membrane space into the matrix couple, in ADP into ATP, to give the cell body energy. On the other hand, enzymes NADPH Oxidase breaks down oxygen into a Superoxide as shown in FIG. 3. A buildup of Superoxide causes loss of energy and cell death. SOD (Superoxide Dismutase) such as that found in the present invention formula, converts Superoxide's to Oxygen (O2) and Water (H2O). To function properly, the preferred SOD requires the mineral ions of Zinc and Copper (Zn, Cu Superoxide Dismutase). Accordingly, the mode of action of the compositions of the present invention is designed to treat several illnesses by targeting mutated cells and promoting healthy cell growth. Further small alterations of the product consisting of the compositions provided by the present invention can be purposed to target a variety of localized pathologies.

The present invented formula contains ionic Zinc and Copper as a Zn, Cu Superoxide Dismutase mimic and may also contain ionic Manganese, Silver Dihydrogen Citrate and ionic Iron for additional effectiveness.

The present invention uses ionic mineral complexes that are capable of penetrating through and being readily absorbed into deep dermal layers of the skin of animal including humans or humans providing an efficient pathway for delivery of vital nutrients to tissue beneath the skin, while having little adverse effect on normal cell operations. Further, ionic mineral complexes are capable of penetrating cell membranes at a rapid pace through simple diffusion.

Following, the formula of a composition for treating mineral deficiencies in creatures, especially in animal including humans, provided by the present invention will be described in detail. The composition includes at least one complex cation and inorganic coordination complexes formed by the coordinate bond formation between an electropositive mineral cation (positive) and molecular groups that have unshared electron pairs. The inorganic coordination complexes include a protective cation mineral ligand carrier that specifically releases the mineral to supply areas of the animal including human that require such a mineral.

The ligand cation complex is absorbed by diffusion into an animal including human and can then move freely throughout the internal animal including human circulation, without the cation binding to targets that do not have mineral deficiency for this cation. The ligand cation complex of the composition is ammonia or super-oxide dismutase. In some embodiment, the ligand cation complex is ammonium hydrogen sulphate ((NH₄) HSO₄). The complex cation of the composition is mineral cation. In some embodiment, the mineral cation is zinc cation or copper cation or zinc and copper cations.

In preference the composition further includes at least one of manganese, magnesium, cobalt, chromium, molybdenum, selenium, and vanadium.

In preference the composition further includes botanical extracts, animal including human hormones, vitamins, and mineral supplements necessary for animal including human health.

The ammonia ligand configuration also acts as an artificial super-oxide dismutase (SOD) which acts as a free oxygen scavenger to alleviate animal including human stress, for example, under drought and mechanical damage.

Testimonials have reported beneficial results in the treatment of insects and worms. These results indicate an extended deterrence against various animal including human issues with no detrimental side effects. These preliminary results suggest that an animal including human with balanced nutrition particularly zinc, copper and sulphur is able to create its own resistances and deterrents. The detailed descriptions for the testimonials will be presented accompany with the treatment effect pictures at last of this specification.

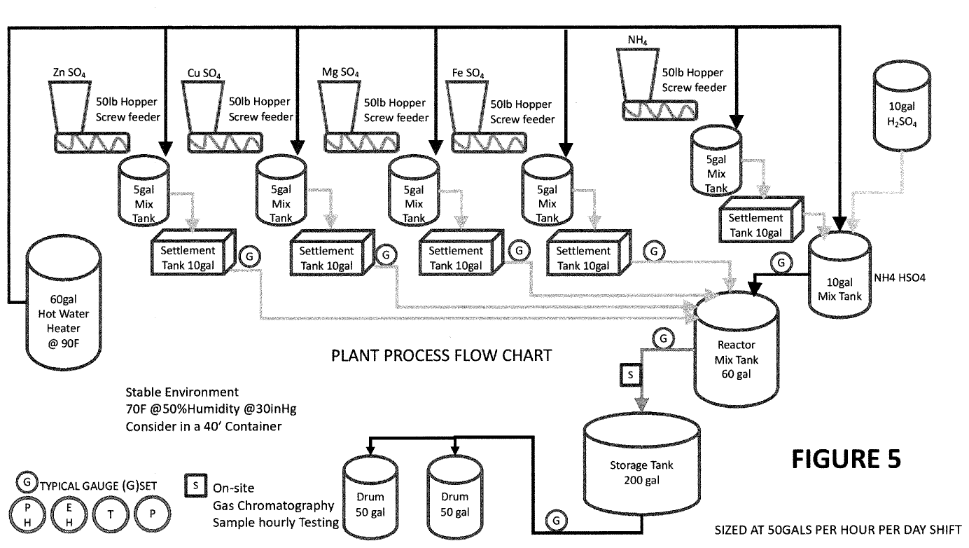

The flow chart in FIG. 5 illustrates the method of production where elements are combined to manufacture the formulation provided by the present invention. This method reduces the heat generated from exothermic reactions when combining these elements.

A method of supplementing nutritional intake of a living animal including human, the method comprises treating an animal including human with a composition, the composition being formed by steps of:
1. preparing a solution of ammonium hydrogen sulphate ((NH₄)HSO₄) using sulfuric acid (H₂SO₄);
2. diluting the ammonium hydrogen sulphate with water to form a mixture;
3. adding sulfuric acid;
4. adding a solution iron sulphate (FeSO₄) and water;
5. adding a solution manganese sulphate (MgSO₄) and water;
6. adding a solution zinc sulphate (ZnSO₄) and water;
7. adding a solution copper sulphate (CuSO₄) and water;
8. agitating the mixture until the ammonium hydrogen sulphate, the sulfuric acid, the water, the manganese sulphate, the iron sulphate, the copper sulphate and the zinc sulphate are blended together to form a zinc metal-ligand complex comprising a first plurality of ammonia ligands and a copper metal-ligand complex comprising a second plurality of ammonia ligands and a manganese metal-ligand complex comprising a third plurality of ammonia ligands and a iron metal-ligand complex comprising a fourth plurality of ammonia ligands.

Consequently, the mixture forms the composition.

In a preferred embodiment, beneficial, symbiotic, supplemental ingredients are to the minerals/acids complex mixture which would increase the efficacy of the final compound. The product is in the form of cream that contains the following ingredients: Deionized Water (Aqua), Mineral Oil, Propylene Glycol, Isopropyl Palmitate, Vegetable Glycerin, Stearyl Alcohol, Cetyl Alcohol, Dimethicone, Steareth-21, Steareth-2, PEG-50 Shea Butter, Vitis Vinifera (Grape) Seed Extract, Co-Q10 Enzyme (Ubiquinone), Tocopheryl Acetate (Vitamin E), Niacinamide (Vitamin B3), Sodium Ascorbyl Phosphate (Vitamin C), Glutathione, Panthenol (Pro-Vitamin B5), Pyridoxin (Vitamin B6), Copper Sulfate (and) Zinc Sulfate (and) Sulfuric Acid, Sodium Hydroxide, Silver Dihydrogen Citrate (and) Citric Acid, Xanthan Gum, Magnesium Aluminum Silicate, Tetrasodium EDTA, Citric Acid, Saccharomyces Lysate Extract, Phospholipids, Lecithin, Sodium Milkamidopropyl PG-Diammonium Chloride Phosphate, Methylparaben, Propylparaben, Diazolidinyl Urea.

In another preferred embodiment, the product is in the form of liquid, which includes the following ingredients: Purified Water (Aqua), Paraffinum Liquidum, Propylene Glycol, Isopropyl Palmitate, Vegetable Glycerin, Stearyl Alcohol, Cetyl Alcohol, Steareth-21, Steareth-2, Tetrahydrodiferuloylmethane (Tetrahydrocurcuminoids), Aloe Barbadense (Aloe Vera) Leaf Juice Extract, Copper Sulfate (and) Zinc Sulfate (and) Ammonium Sulfate (and) Sulfuric Acid, Sodium Hydroxide, Silver Dihydrogen Citrate (and) Citric Acid, Tetrahexyldecyl Ascorbate (Vitamin C), Grapeseed Extract (Vitis Vinifera), Co-Q10 Enzyme (Ubiquinone), Tocopherol Acetate (Vitamin E Acetate), Arnica Montana (Arnica Flower) Extract, Glutathione, Panthenol (Pro-Vitamin B5), Pyridoxin (Vitamin B6), Superoxidase Dismutase, Xanthan Gum, Magnesium Aluminum Silicate, Tetrasodium EDTA, Citric Acid, Saccharomyces Lysate Extract, Phospholipids, Lecithin, Methylparaben, Propylparaben, Diazolidine Urea.

In some preferred embodiments, the compositions include a high level of sulfur in the form of sulfate which functions as an active secondary transport complex. This sulfate has a negative charge and is not solvated or wrapped up in transport proteins or ligands like positive zinc and copper ions. Movement of sulfate into the cell is dependent on a number of factors including concentrations of other ions, pH and concentration gradients across the membrane. If influx of sulfate is favored it can aid in transport of copper and zinc ions in their movement into the cell. This is especially useful when the active ATP transport process is inhibited. This sulfur complex can operate synergistically with the mineral complex by accelerating absorption of affected cells. The high level of free sulfur can be transported to various locations and may speed reconstitution of damaged tissue affected by disease.

Sulfur is a non-metallic acidic micro-mineral usually consumed as part of a larger zinc, copper compound whose expression is not usually considered an aid to mitigation of aberrant diseased cells. However, the benefits of sulfur are well known and a formulation that combines a high-sulfur content (NH₄HSO₄) base with minerals that contain sulfur (zinc sulfate, etc.) provides an abundance of free sulfur that may accumulate in those regions of the human body requiring attention.

The preferred mineral would be a sulfate for that reason but is not necessary for success of free sulfur. Benefits of sulfur include boosting the immune system and providing pain relief to targeted cells. The mechanism by which free sulfur produced in this mixture operates similarly to the operative mode of glucosamine sulfate, chondroitin sulfate, and dimethyl sulfoxide.

In preference, the method of manufacturing the composition further comprising: adding at least one delivery agent to the composition prior to the step of treating. The delivery agent selected from the group consisting of a surfactant, dimethyl sulfoxide, a urea-based compound, a detergent, a hygroscopic compound, and combinations thereof.

In preference the ratio of zinc to copper of the composition is 7:2.

In preference the composition has a pH of less than 1.0.

In preference the method of manufacturing the composition further comprising a step of analyzing the animal including human to determine a specific nutrient required and, wherein the composition further includes a nutrient matched to the specific nutrient required.

In preference the method of manufacturing the composition further comprising adding at least one of manganese, magnesium, cobalt, chromium, molybdenum, selenium, and vanadium into the composition.

In preference the method of manufacturing the composition further comprising adding at least one of an animal including human hormone, an auxin, or an animal including human extract into the composition.

In a further form of the invention there proposed a method of supplementing bio-mineral nutritional intake of a healthy, living animal including human, the method comprising steps of treating a healthy animal including human with a composition, the composition being formed by:
1. preparing a solution of ammonium hydrogen sulphate ((NH₄)HSO₄) using sulfuric acid (H₂SO₄);
2. diluting the ammonium hydrogen sulphate with water to for a mixture;
3. adding solutions of manganese sulphate and water, iron sulphate and water, zinc sulphate (ZnSO₄) and water, copper sulphate (CuSO₄) and water to the mixture, wherein the ratio of zinc to copper to manganese to iron is 7:2:1:1;
4. agitating the mixture until the ammonium hydrogen sulphate, the sulfuric acid, the water,
   the solution of copper sulphate and the solution of zinc sulphate are blended together to form a zinc metal-ligand complex comprising a first plurality of ammonia ligands and a copper metal-ligand complex comprising a second plurality of ammonia ligands;
5. further dilution of the mixture with water may be required to form the composition, wherein the composition has a pH of less than 1.0.

The present invention also discloses the composition is applied for treating the healthy animal including human comprises a topical application (cream or spray), injection, irrigation or oral.

The step of treating the healthy animal including human comprises adding the composition to a topical application to the affected area of the animal including human. Upon introduction the animal including human, the composition bonds with a super oxide dismutase composition and is transported to mineral-deficient parts of the animal including human.

The product formulation as manufactured provides a delivery system for moving mineral ions to the mineral deficient areas in animal including humans using highly bioavailable cations through a complex ligand system. Ammonia ligands form a bond with the free metallic ions, especially zinc and copper to regions of the animal including human that require such minerals. Other minerals to be named follow the same pattern of dispersal throughout the animal including human in a systemic nature. The animal including human's natural metabolism will discard excess metals as described in the metabolic pathway's descriptions although an over-whelming amount of the composition is possible and may cause necrosis in the animal including human. The ionic nature of the formulation allows easy access to areas of the animal including human with deficiencies and provides a formulation that is stress relieving. Systemic delivery of targeted, and minerals create more robust animal including humans. Benefits to animal including humans accomplished organically, without the use of additive or subtractive genetic modify-techniques and is therefore considered non-GMO. Significantly smaller quantities of additive minerals and growth compounds compared to conventional husbandry. Organic formula and dilute application requirements should substantially reduce economic, social, ecological, and regulatory concerns associated with runoff into water sources. Use of less water compared to conventional farming methods. The ability to deploy self-contained animal including human growth systems for use in hostile environments such as the polar regions, deserts. The disclosure further provides a method of reducing the time to maturity comprising supplementing nutritional intake in a living animal including human by the disclosed method. The disclosure further provides a method of increasing farm yields for food comprising supplementing nutritional intake in a living animal including human by the disclosed method. The disclosure further provides a method of creating more robust animal including humans by systemic delivery superior to current technologies.

The present disclosure relates to a method of supplementing nutritional intake in a living animal including human, the method comprising steps of: treating an animal including human with a composition, the composition being formed by: adding ingredients of water, H₂SO₄, (NH₄) of targeted nutrients and minerals to crops comprising supplementing nutritional intake of a living animal including human by the disclosed method. The disclosure further provides a method of increasing the nutritional quality of animal including human derived produce, including the SO₄, copper and zinc into a mixture; agitating the mixture until the ingredients are blended together; diluting the mixture with water to form the composition.

The present disclosure relates to a method of supplementing nutritional intake of a healthy, living animal including human, the method comprising steps of: treating a healthy animal including human with a composition, the composition being formed by: adding ingredients of water, H₂SO₄, (NH₄)₂SO₄, copper and zinc into a mixture; agitating the mixture until the ingredients are blended together; diluting the mixture with water.

It has been surprisingly found that the disclosed methods, which utilize a bioavailable mineral composition comprising water, H₂SO₄, (NH₄)₂SO₄, copper and zinc leads to many positive effects in animal including humans, for example healthy animal including humans, and in particular in food crop animal including humans. Animal including humans treated according to the disclosed method have been found to be faster growing, healthier and/or more robust. Additionally, the disclosed method may allow for fewer applications of the treatment compared to other known mineral treatments leading to considerable cost and time savings. The benefits of the disclosed methods for supplementing nutritional intake in living animal including humans may be summarized as including one or more of improvements:
Improved fertility
Systemic delivery of targeted nutrients and minerals to animal including humans to create more robust animal including humans
Ability to increase the nutritional balance of animal including human
Benefits to animal including humans accomplished organically, without the use of additive or subtractive genetic modification techniques and is therefore considered non-GMO
Significantly smaller quantities of additive minerals and growth compounds compared to conventional treatments
Organic formula and dilute application requirements should substantially reduce economic, social, ecological, and regulatory concerns

The disclosure further provides a method of reducing the time to supplement nutritional intake of a living animal including human by the disclosed method.

The disclosure further provides a method of increasing food supplementing nutritional intake of a living animal including human by the disclosed method.

The disclosure further provides a method of creating more robust animal including humans by systemic delivery of targeted nutrients and minerals comprising supplementing nutritional intake of a living animal including human by the disclosed method

The disclosure further provides a method of increasing the nutritional quality of animal including human food sources, including the potential to engineer nutrition into our food, supplementing nutritional intake by the disclosed method.

The disclosure further provides a method of using less water when supplementing nutritional intake of a living animal including human by the disclosed method.

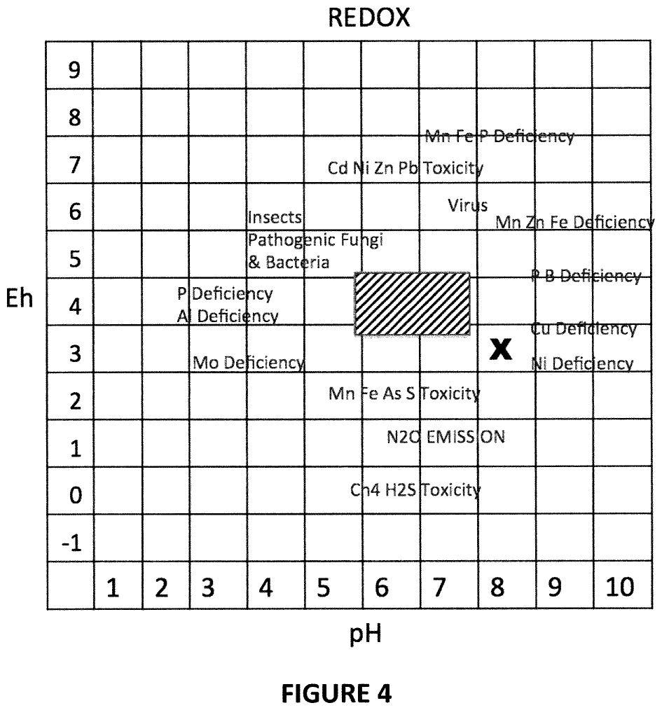

As shown in FIG. 4, the relationship of Electrical Charge (Eh) to Acid-Alkalinity (pH) of REDOX. The shaded box area is the general sweet spot for optimal animal including human development. This varies with specific animal including humans. Also, viruses have a preferred sweet spot. By using the patented solution, we adjust the redox in an animal including human to the sweet spot whereby the animal including human is able to mitigate viruses.

The manufacturing process described below produces a complex having ammonia ligand bonds with specific cations. The cations are carried by the ligand bonds and protected from being immediately bonded with the first available negative ions thus enabling free movement between and within the cells of the animal including human. Each of the minerals will be processed in the acid/base solution resulting in products that have a high acidity value, yet not being corrosive to living tissue. A noticeable concentration of reactive ammonia is also produced by the acid/base reaction. Complex cations and inorganic coordination complexes are formed that can move the cations in a relatively stable fashion and allow transport throughout the animal including human.

A solution containing a mix of the prepared mineral(s) may contain only one of the minerals and additional supplements or all the mentioned minerals plus selected supplements to achieve the desired effect. For example, a zinc deficiency may only require zinc and a small amount of copper to balance (Mineral Inter-relationships) the effect of the possibility of too much zinc and other supplements (urea for example) to reinforce the effect of the mineral. All the minerals will act independent of one another much as gases do in Dalton's Law and be assimilated by the animal including human on an as-needed basis. For example, copper and zinc can be antagonistic to one another in an animal including human, but the two minerals can be put into the formulation at the right proportions and counter act the influence of one against the other. In some embodiments of the present invention, the copper and zinc also act to counterbalanced each other physiologically on a basis of 7:2 mole ratio (zinc to copper) and with the ammonia (NH3) form the composition the subject of this invention, a form of ligand complex. The ligand travels through the selective membrane (epidermis) and travels through the animal including human's xylem and/or phloem to a mineral deficient tissue where a physiological change will occur, and the minerals are un-encapsulated from the ligand complex and are usable as part of the metabolic pathway in the animal including human.

The ligand formation is obtained during manufacture. However, the use of the ionic mineral in the formulation can be utilized to form an artificial superoxide dismutase (SOD). Animal including humans normally form SODs however since silicon is necessary in most animal including humans and is to support the structure. An example of incorporation of the composition as described with reference to the superoxide dismutase (SOD) cycle will use copper (Cu) and zinc (Zn). The composition will be incorporated into the SOD on an as needed basis and attached to the mineral complex to make a Cu-SOD, a Zn-SOD, or a Cu/Zn-SOD. The enzyme superoxide dismutase catalyzes dismutation of super oxide into oxygen and hydrogen peroxide. Therefore, it is an important antioxidant defense in almost all cells exposed to oxygen. The SOD catalyzed dismutation of super oxide may be written using the following half reactions:

M(“+¹)+SOD+O²-M(“●)+SOD+O

M(+)+SOD+O²+2H+-M(“+¹)+SOD+H₂O₂

Where M may be, but is not limited to:

(a)Cu(n=1);(b)Zn (n=2);(c)Mn(n=2);Fe(n=2);(e)Ni(n=2).

In this reaction the oxidation state of the metal cation oscillates between n and n+1. Several common forms of SOD 55 exist and are proteins co-factored with copper (Cu) and zinc (Zn), manganese (Mn), iron (Fe) or nickel (Ni). Cytosols of almost all eukaryotic cells contain SODs and combine with copper and zinc (Cu—Zn-SOD)s. The Cu—Zn SOD and design is a homodimer of molecular weight of approximately 32,500. The Cu and Zn are joined primarily by hydrophobic and electrostatic interactions. The ligand complexes of copper and zinc are histidine side chains whereas the ligand complexes of manganese ions are three histidine side chains. The incorporation of the ligand complex into the SOD enables the ligand complex to travel throughout the animal including human in a protected form and without compromising the effectiveness of the ligand complex. Once the SOD with the ligand complex reaches a target cell the cat-ion within the ligand complex is released into the cell.

The resulting formulation can be prepared in many ways for application and will vary with the intended use. Formulations prepared for treatment for animal including human mineral deficiency will require selection of the proper formulation containing the necessary minerals. The formulations are stable at a pH near or below pH 1.0 in a wide variety of carriers. However, the active composition is prepared using the liquid. Additionally, the low pH of the composition may be diminished if the composition crystallizes.

The mineral cation ingredients in the active composition will vary in proportion depending on the intended use and be added in certain formulation depending on the type and purpose thereof. The added inert ingredients used in the formulation will also vary considerably depending on the site and purpose of the application. Other active compounds may be added if the proposed components prove to be beneficial to the formulation. However, the basic ingredients are known to be effective without any additional components.

Additional elements may be added to the composition that aid in the overall effectiveness of the formula. For example, animal including human extracts and urea have demonstrated additional effectiveness for certain animal including human groups. Auxins and animal including human hormones may be useful for specific uses.

Treatment of mineral deficiencies and/or promoting general health of the animal including human may be carried out by topical application. The composition can also be applied as an injectable in cases where there is a special effort to incorporate minerals into an animal including human to ensure absorption. The animal including human may be soaked, sprayed, drenched or submerged in a diluted solution (the ratio of the composition to the water is 1:2 and above). A newborn animal including human maybe treated by spraying, misting, drenching directly. The formulation is systemic to the dermal surface of the skin.

The formulation can also be applied in a food source with the formulation being applied to the animal including human. The formulations can also be incorporated into creams, gels, sprays, mists, agars and dermal and transdermal patches.

The bioavailable minerals for the mitigation of viruses in animal including humans provided by the present invention has a significant and positive effect on reduction of pathogen attack by increasing the animal including human's responses by discouraging a pathogen's preference as a reproduction environment. It aids farmers with animal including human husbandry with early immune development in animal including human offspring. It may accelerate maturation rate of young animal including humans. It also aids animal including humans with recovery from pathogen attack. With optimal concentration, it can be customized to fit the critical need of the animal including human, balancing development with production of immunity and mitigating pathogen attack.

This invention reduces the level of Superoxide's in cells which when over produced, due to extreme stress may stimulate ROS (Reactive Oxygen Species) which kills healthy cells. By preventing the over production of the Superoxide's and ROS, the invented formula can thereby stimulate healthy cells and prevent various viruses from reproducing.

Other benefits of the products manufactured by the method of the present invention include, highly absorbable, precisely targeted application to problem infection sites, abnormal skin growths or discolorations, efficiently delivery of concentrates of active ingredients to targeted skin tissue and promoting health skin renewal to all the skin layers.

Some testimonials prove the effects of the products made from the compositions of the present invention will be illustrated in the following.

Generally, it has become unacceptable to evaluate technology on animal including humans. Consequently, our studies have been conducted on consenting adults, and because of the non-invasive application potential side effects mitigated. Additionally, results are delivered without the need for speculation and interpretation. Testimonials have reported beneficial results in the topical application of the product namely Sundancer Topical. These results indicate an extended deterrence against various virus issues.

These preliminary results suggest that an animal including human with balanced nutrition particularly Zinc, Copper and Sulphur is able to create its own resistances and deterrents.

Frank Berry, Palos Verdes, Calif. (310-720-9492); Jennifer (40+ year old female) in Phoenix Ariz. I have MS plus I am somewhat overweight and because of these conditions, when I got Covid19

My doctor was very concerned. My father sent me a product from Sundancer Products called “Sun-FROST SSR”. I applied it to my nose, mouth, throat and chest 3 times/day and within 3 days my symptoms were gone, and my taste & smell returned.

Shayna & Branden Butyrin, (40+ year old female and male) Indio, Calif. (480-510-7663);

Branden and I both were diagnosed with COVID 19 as well as several other people who attended a party however, because as a treatment, we used Sundancer's Sun-FROST on our face, neck and chest, immediately after we were diagnosed, our symptoms were very mild and were completely gone in less than a week. The other people who had contacted the disease were very sick for more than a month. I am convinced that the product made the difference.

1. A composition for treating mineral deficiencies, comprising:
at least one complex cation and inorganic coordination complexes formed by the coordinate bond formation between an electropositive mineral cation and molecular groups;
a. the inorganic coordination complexes including a protective cation mineral ligand carrier, that specifically releases the mineral cation to supply areas that require such a mineral;
b. wherein the complex cation and inorganic coordination complexes is absorbed by diffusion and the mineral cation can then move freely throughout the internal animal or human circulation, without the cation binding to targets that do not have mineral deficiency for this cation.

2. The composition of claim 1, wherein the ligand cation complex is ammonia or super-oxide dismutase.

3. The composition of claim 1, wherein the ligand cation complex is ammonium hydrogen sulphate ((NH₄) HSO₄).

4. The composition of claim 1, wherein the complex cation is mineral cation.

5. The composition of claim 4, wherein the mineral cation is zinc or/and copper cation.

6. The composition of claim 1, further comprising one of manganese, magnesium, cobalt, chromium, molybdenum, selenium, Silver Dihydrogen Citrate and vanadium.

7. The composition of claim 1, further comprising one of botanical extracts, animal including human hormones, vitamins, and mineral supplements necessary for animal including human health.

8. The composition of claim 1, wherein the ratio of zinc to copper of the composition is 7:2.

9. The composition of claim 1, wherein the composition has a pH of less than 1.0.

10. A composition of treating for disease using a nasal spray or nebulizer comprising the composition of any one of preceding claims, wherein further comprises Saline Solution, the Cu/Zn complex, Silver Dihydrogen Citrate, and hydrogen peroxide.

11. A method of manufacturing a composition for treating mineral deficiencies of claim 1, comprising:
preparing a solution of ammonium hydrogen sulphate ((NH4) HSO₄) using sulfuric acid (H₂SO₄);
diluting the ammonium hydrogen sulphate with water to form a mixture; adding sulfuric acid;
adding a solution iron sulphate (FeSO4) and water; and/or
adding a solution manganese sulphate (MgSO4) and water; and/or
adding a solution zinc sulphate (ZnSO4) and water; and/or
adding a solution copper sulphate (CuSO4) and water; and
agitating the mixture until the ammonium hydrogen sulphate, the sulfuric acid, the water, the manganese sulphate, the iron sulphate, the copper sulphate, and the zinc sulphate are blended together to form a zinc metal-ligand complex comprising a first plurality of ammonia ligands and a copper metal-ligand complex comprising a second plurality of ammonia ligands.

12. The method of claim 10, further comprising:
adding at least one delivery agent to the composition prior to the step of treating.

13. A method of treating mineral deficiencies comprising:
treating a creature with a composition, the composition being formed by steps of claim 10.

14. The method of claim 12, wherein the creature is an animal including human.
